(19) United States
(12) Patent Application Publication
(10) Pub. No.: US 2026/0157782 A1
(43) Pub. Date: Jun. 11, 2026

(54) ABLATION CATHETER GUIDEWIRE

(71) Applicant: Medtronic, Inc., Minneapolis, MN (US)

(72) Inventors: Mark T. Stewart, Lino Lakes, MN (US); Timothy G. Laske, Shoreview, MN (US); Richard J. Olson, Blaine, MN (US); Daniel C. Sigg, St. Paul, MN (US); Lars M. Mattison, Blaine, MN (US)

(21) Appl. No.: 19/127,151

(22) PCT Filed: Nov. 7, 2023
(86) PCT No.: PCT/US2023/078956

§ 371 Date: May 5, 2025

Related U.S. Application Data
(60) Provisional application No. 63/382,842, filed on Nov. 8, 2022.

(51) Int. Cl. A61B 18/00; A61B 90/00; A61M 25/09

(52) U.S. Cl. CPC A61B 18/00; A61B 90/39; A61M 25/09; A61B 2018/00077; A61B 2018/00083; A61B 2018/00178; A61B 2018/00577; A61B 2018/00839; A61B 2090/3966; A61M 2025/09075; A61M 2025/09108; A61M 2025/09133; A61M 2025/09166; A61M 2025/09175; A61M 2205/0233; A61M 2205/32; A61M 2207/00; A61M 2230/04

(57) ABSTRACT

A guidewire includes a core member including a proximal portion having a first stiffness and a distal portion having a second stiffness less than the first stiffness. The guidewire further includes an electrical insulator encapsulating the distal portion of the core member and at least a portion of the proximal portion of the core member. The electrical insulator comprises a material configured to electrically insulate the core member from pulsed field ablation (PFA) energy delivered by a PFA catheter guided by the guidewire. The guidewire further includes electrode positioned along the distal portion.

This application claims the benefit of U.S. Provisional Patent Application 63/382,842, filed 8 Nov. 2022, the entire content of which is incorporated herein by reference.

### TECHNICAL FIELD

The present technology is related to ablation catheters. In particular, various examples of the present technology are related to assisting ablation using ablation catheter delivery devices.

### BACKGROUND

Tissue ablation is a medical procedure commonly used to treat conditions such as cardiac arrhythmias, which includes atrial fibrillation. For treating cardiac arrhythmias, ablation can be performed to modify tissue, such as to stop aberrant electrical propagation and/or disrupt aberrant electrical conduction through cardiac tissue. Ablation techniques include pulsed field ablation (PFA), cryoablation, laser ablation, radioablation and radiofrequency (RF) ablation.

Cardiac arrhythmias are a group of conditions that cause an irregular heartbeat or conduction pattern. Ablation may be used to create a safe and effective lesion or set of lesions at the origin of the irregular heartbeat or in regions that aid in the termination of arrhythmias without causing damage to adjacent structures or surrounding tissue, ideally resulting in no need for a maintenance treatment regimen, such as medications or cardioversions.

### SUMMARY

The present technology is directed to devices, systems, and methods for assisting ablation using ablation catheter delivery devices. An ablation catheter delivery device may comprise an insulated guidewire configured for use with relatively high voltage PFA energy delivery electrodes. The guidewire may include one or more of: navigation electrodes, mapping electrodes, and/or ablation electrodes.

A standard guidewire may have very little electrical insulation and if it should become prolapsed, e.g., fold back over on itself and/or the ablation catheter and come in close proximity to the PFA energy delivery electrodes, the result may be a short circuit of high energy passing between adjacent PFA electrodes. Such a short circuit may be undesirable. In accordance with the devices, systems, and techniques herein, a guidewire may be configured to reduce a probability of such a short circuiting, and may provide additional features and/or capabilities.

For example, a guidewire may have varying stiffness along its length to balance pushability of the guidewire with flexibility for navigating the guidewire within the patient while preventing perforating tissue, e.g., a vessel wall, and preventing electrically shorting PFA electrodes. The guidewire may have a varying stiffness along its length to provide adequate stiffness at a proximal portion for manipulation and navigation of a transition and distal portions having relatively more flexibility for improved and relatively atraumatic passage within the patient, e.g., through vasculature. The guidewire may include a core having a first thickness at the proximal portion, and second and smaller thickness at the distal portion, and a thickness that tapers between the two thicknesses at the transition portion. The core may be insulated within a surrounding dielectric material. The guidewire may include an insulative jacket surrounding the core and dielectric material and configured to house signal wires and/or conductors electrically coupled to the navigation, mapping, and/or ablation electrodes positioned along the transition and/or proximal portions. In this way, the guidewire of this disclosure is less likely to prolapse, and therefore less likely to short circuit.

In some examples, at least a portion of the guidewire may be lubricious and/or have a lubricious coating and/or treatment on at least a portion of the outer surface of the guidewire. For example, a distal portion and a transition portion of the guidewire may have a hydrophobic coating, e.g., a polymer, silicone, polytetrafluoroethylene (PTFE), or the like, to increase lubricity. In some examples, the distal and transition portions of the guidewire may have a hydrophilic coating to reduce friction during deployment and for easier movement in tortuous vessels. In some examples, at least a portion of the guidewire may include one or more visibility features, such as a radiopaque marker or feature visible via fluoroscopy, a navigation coil, or the like.

In one example, this disclosure describes a guidewire including: a core member including: a proximal portion having a first stiffness; and a distal portion having a second stiffness less than the first stiffness; an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter guided by the guidewire; and an electrode positioned along the distal portion.

In another example, this disclosure describes a medical system including: a pulsed field ablation (PFA) catheter; and a guidewire including: a core member including: a proximal portion having a first stiffness; and a distal portion having a second stiffness less than the first stiffness; an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter; and an electrode positioned along the distal portion.

In another example, this disclosure describes method of forming a guidewire including: encapsulating a distal portion of a core member and at least a portion of a proximal portion of the core member within an electrical insulator, wherein the electrical insulator comprises a material configured to electrically insulate the core member from pulsed field ablation (PFA) energy delivered by a PFA catheter, wherein the proximal portion of the core member has a first stiffness and the distal portion of the core member has a second stiffness less than the first stiffness; and positioning an electrode along the distal portion.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example system for delivering ablation.

FIG. 2 is a conceptual diagram illustrating an example guidewire for guiding an ablation catheter.

FIG. 3 is a cross sectional view of a portion of the example guidewire of FIG. 2.

FIG. 4 is a flow diagram illustrating an example method of forming a guidewire for guiding an ablation catheter.

FIG. 5 is a block diagram illustrating an example controller of an ablation system, in accordance with one or more aspects of this disclosure.

### DETAILED DESCRIPTION

Over-the-wire devices that are designed to deliver Pulsed Field Ablation (PFA) using high voltages can encounter issues when standard wire coil type guidewires are used A standard guidewire may have very little electrical insulation and if it should become prolapsed, e.g., fold back over on itself, and/or the PFA catheter, and come in close proximity to the PFA energy delivery electrodes, the result may be a short circuit of high energy passing between adjacent PFA electrodes. In addition, such standard guidewires have no individual electrodes mounted on them that would allow diagnostic mapping of intracardiac electrograms or delivery of diagnostic pacing in the heart, or delivery of PFA, e.g., of a lower voltage and/or PFA dose than a PFA catheter guided via the guidewire. A standard guidewire may also be difficult to track on cardiac navigation systems and may require the physician to use X-ray fluoroscopy to visualize the location of the guidewire.

In accordance with the devices and techniques disclosed herein, an example guidewire includes a core member comprising a proximal portion having a first stiffness, e.g., configured to allow a user to manipulate the guidewire, and a distal portion having a second stiffness that is less than the first stiffness, e.g., configured to be atraumatic and with sufficient flexibility to navigate the guidewire with the patient, e.g., through tortuous vasculature. The guidewire further includes an insulator encapsulating the distal portion of the core member and at least a portion of the proximal portion of the core member, e.g., a dielectric material configured to insulate the core member from relatively high voltage PFA energy (e.g., at least 1,500 volts) delivered by a PFA catheter using the guidewire to navigate to target tissue.

In some examples, the guidewire may further include an electrode positioned and/or disposed along the distal portion of the core member. The electrode may be external to the insulator, and may be electrically connected to a conductor and/or signal wire running along the longitudinal length of the guidewire to connect the electrode to a controller and/or sensor. In some examples, the electrode may be electrically connected to the core member, e.g., the core member may be electrically conductive and may be configured to function as a signal wire. The guidewire may further include a jacket surrounding the insulator and configured to house the conductor and/or signal wire, e.g., the jacket may encapsulate the signal wire and insulated core member to electrically insulate the signal wire from electrical contact with PFA electrodes of the PFA catheter, In some examples, the electrode may be radiopaque and useful for aiding in guiding the guidewire during insertion of the guidewire to target tissue. In some examples, the electrode may be configured to deliver PFA energy, e.g., a high voltage pulsed electric field. For example, the electrode may be configured to deliver a relatively lower “dose” and/or amount of PFA energy relative to the PFA electrodes of the PFA catheter so as to provide additional ablation control, e.g., to “fine tune” the ablation. In some examples, the electrode may be configured to sense a signal, such as an intracardiac electrocardiogram (EGM) signal which may be useful for determining positioning of the guidewire relative to target tissue and/or relative to the PFA catheter, e.g., a distal end of the PFA catheter.

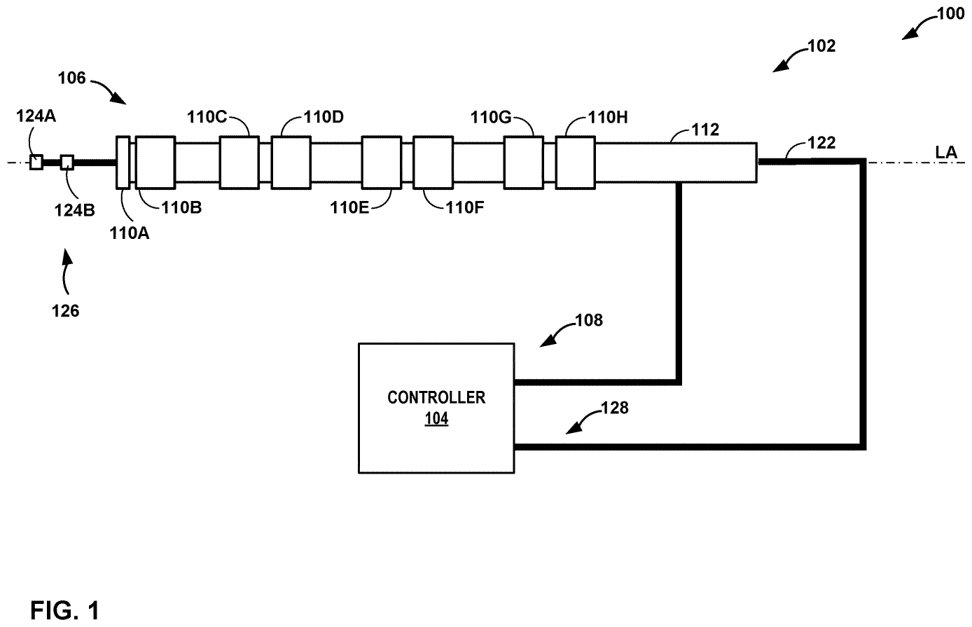

FIG. 1 is a conceptual diagram illustrating an example system 100 for delivering ablation. System 100 includes a catheter 102, a controller 104, and guidewire 122. In general, to deliver ablation, a practitioner (e.g., electrophysiologist, interventional cardiologist, etc.) may insert guidewire 122 into a patient and navigate the guidewire to a target tissue site within the patient. The practitioner may then insert one or more of catheter 102 into the patient and guide the catheter 102 to the target site via the guidewire. The practitioner may then cause controller 104 to deliver, via catheter 102, energy (e.g., PFA energy, radiofrequency ablation energy, laser ablation, radio ablation, cryoablation energy, or the like) to target tissue of a patient. Ablation may cause lesions in target cardiac tissue which may mitigate or stop cardiac arrhythmias. In some examples, controller 104 may cause catheter 102 to deliver electroporation energy, e.g., PFA energy. Electroporation may be a phenomenon causing cell membranes to become hyperpermeable (that is, permeable for molecules for which the cell membrane may otherwise be impermeable or semipermeable). Electroporation, which may also be referred to as electropermeabilization, pulsed electric field treatment, non-thermal irreversible electroporation, irreversible electroporation, high frequency irreversible electroporation, nanosecond electroporation, or nanoelectroporation, may involve the application of high-amplitude pulses to cause physiological modification (i.e., permeabilization) of the cells of the tissue to which the energy is applied. These pulses may be short (for example, nanosecond, microsecond, or millisecond pulse width) in order to allow the application of high voltage, high current (for example, 20 or more amps) without long duration(s) of electrical current flow that may otherwise cause significant tissue heating and muscle stimulation. The pulsed electric energy may induce the formation of microscopic defects that result in hyperpermeabilization of the cell membrane. Depending on the characteristics of the electrical pulses, an electroporated cell can survive electroporation, referred to as “reversible electroporation,” or die, referred to as “irreversible electroporation” (IRE). Reversible electroporation may be used to transfer agents, including genetic material and other large or small molecules, into targeted cells for various purposes, including the alteration of the action potentials of cardiac myocytes.

Catheter 102 may include elongated structure 112 carrying a plurality of energy delivery elements 110A-110H (collectively “energy delivery elements 110”). An energy delivery element may include an electrode (e.g., in the case of a PFA catheter), a cryogenic element (e.g., in the case of a cryoablation catheter), a radiofrequency element (e.g., in the case of a radiofrequency ablation catheter), or another energy delivery element. While the techniques of this disclosure are applicable to any ablation catheter, the example of FIG. 1 is directed to a PFA catheter. Catheter 102 may generally include features that enable insertion of catheter 102 into a patient and navigation of catheter 102 to a target tissue site. Elongated structure 112 may include a distal portion 106 and a proximal portion 108. Energy delivery elements 110 may be generally positioned at distal portion 106, while proximal portion 108 may be connected to controller 104. Energy delivery elements 110 may be of any suitable geometry. Example geometries of electrodes include, but are not necessarily limited to, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes). Energy delivery elements 110 may be axially distributed along longitudinal axis LA of elongated structure 112 or in several other configurations. In some examples, catheter 102 may include one or more energy delivery elements 110 and the geometry of the one or more energy delivery elements 110 may include a balloon, which may be inflated when performing ablation and deflated when navigating catheter 102 to the target tissue. The delivery elements 110 may also be in a circular form, in an array, along multiple splines, or in other configurations.

Elongated structure 112 may include conductors configured to carry electrical signals between energy delivery elements 110 and controller 104. In some examples, elongated structure 112 may include a separate conductor for each of energy delivery elements 110. For instance, in the example of FIG. 1 where energy delivery elements 110 includes eight electrodes, elongated structure 112 may include eight separate conductors. In this way, elongated structure may enable each electrode of energy delivery elements 110 to be driven with a different signal from controller 104. In other examples, multiple electrodes of energy delivery elements 110 may share a common conductor. For instance, energy delivery elements 110C and 110D may be connected to a same (e.g., a common) conductor. While such a common conductor arrangement may reduce energy delivery element flexibility (e.g., as electrodes connected to the common conductor may be driven with a same signal), such an arrangement may reduce manufacturing complexity and/or cost and may increase the structural flexibility of catheter 102.

As shown in FIG. 1, energy delivery elements 110 may include a tip electrode (e.g., energy delivery element 110A), which may be a ring electrode with a “cap” covering at least a portion of a tip of elongated structure 112. In some examples, the tip electrode may be chamfered or otherwise rounded (e.g., to enable easier passage of catheter 102 through anatomy of the patient). Energy delivery elements 110 may include a ring electrode (e.g., energy delivery element 110B) that is adjacent to the tip electrode. This ring electrode may be separated (axially along LA) from the tip electrode. Energy delivery elements 110 may include one or more pairs of ring electrodes. A pair of ring electrodes may include two adjacently closely spaced electrodes of energy delivery elements 110. For instance, in the example of FIG. 1, energy delivery elements 110C and 110D may form a first pair of ring electrodes, energy delivery elements 110E and 110F may form a second pair of ring electrodes, and energy delivery elements 110G and 110H may form a third pair of ring electrodes. In general, the first pair of ring electrodes (i.e., energy delivery elements 110C and 110D) may be accompanied by one or more additional electrodes. The one or more additional electrodes may include any combination of pairs of ring electrodes and coil electrodes (e.g., electrodes that include conductors that spiral around elongated structure 112).

In the example of FIG. 1, energy delivery elements 110 are illustrated as has having a larger diameter than elongated structure 112. In some examples, one or more of energy delivery elements 110 may have a diameter that is approximately equal to or less than the diameter of elongated structure 112. For instance, energy delivery elements 110 may be recessed in elongated structure 112 such that the combination results in a relatively smooth outer surface.

Controller 104 may include an energy generator configured to provide electrical pulses to energy delivery elements 110 (or to control the delivery of radio frequency energy or cryogenic energy by energy delivery elements 110) to perform an ablation procedure to cardiac tissue or other tissues within the patient's body, such as renal tissue, airway tissue, and organs or tissue within the cardiac space or the pericardial space. For instance, the energy generator may be configured and programmed to deliver pulsed, high-voltage electric fields appropriate for achieving desired pulsed, high-voltage ablation, e.g., PFA, “pulsed electric field ablation,” and/or pulsed radiofrequency ablation. In some examples, the energy generator may be configured and programmed for achieving desired cryogenic ablation.

Guidewire 122 may include an elongated member configured to be inserted into a patient to a target tissue site and to guide catheter 102 to the target tissue site. For example, catheter 102 may include a lumen configured to receive guidewire 122 and allow catheter 102 to be advanced within the patient along guidewire 122 to the target tissue site. As guidewire 122 may be used with catheter 102, the elongated member of guidewire 122 may be a core member encapsulated within an electrical insulator configured to electrically insulate the core member from electrical energy, such as PFA energy, delivered by energy delivery elements 110. In some examples, the core member may comprise stainless steel, nickel titanium (e.g., Nitinol), a polymer, an additional polymeric stiffening structure, or any suitable material configured to be inserted into a patient, advanced within the patient to a target tissue site, and guide catheter 102. In some examples, the insulator may be configured to electrically isolate the core member from voltages of at least 1,500 volts, or at least 2,000 volts, or at least 3,000 volts, or at least 4,000 volts, or at least 6,000 volts, or at least 8,000 volts, e.g., and the insulator may be configured to do so without dielectric breakdown of the insulator. For example, the insulator may comprise a dielectric material having a resistivity, dielectric strength, and thickness configured to electrically isolate the core member from voltages of at least 1,500 volts. For example, the insulator may comprise a polyimide having a coating thickness of between about 1 micrometer and 500 micrometers, between about 10 micrometers and 50 micrometers, or between about 10 micrometers and about 30 micrometers.

Guidewire 122 may include and/or carry one or more electrodes, such as electrodes 124A and 124B (collectively, “electrodes 124”). Electrodes 124 may be conductors configured to sense electrical current and/or fields and/or as energy delivery elements (e.g., PFA delivery elements), radiofrequency elements, or other energy delivery elements. Electrodes 124 may be configured to be energy delivery elements configured to delivery cryogenic energy. In some examples, electrodes 124 may be substantially similar to energy delivery elements 110, however, smaller in size and/or energy delivering capability. Guidewire 122 may include a distal portion 126 and a proximal portion 128. Electrodes 124 may be generally positioned at distal portion 126, while proximal portion 128 may be connected to controller 104.

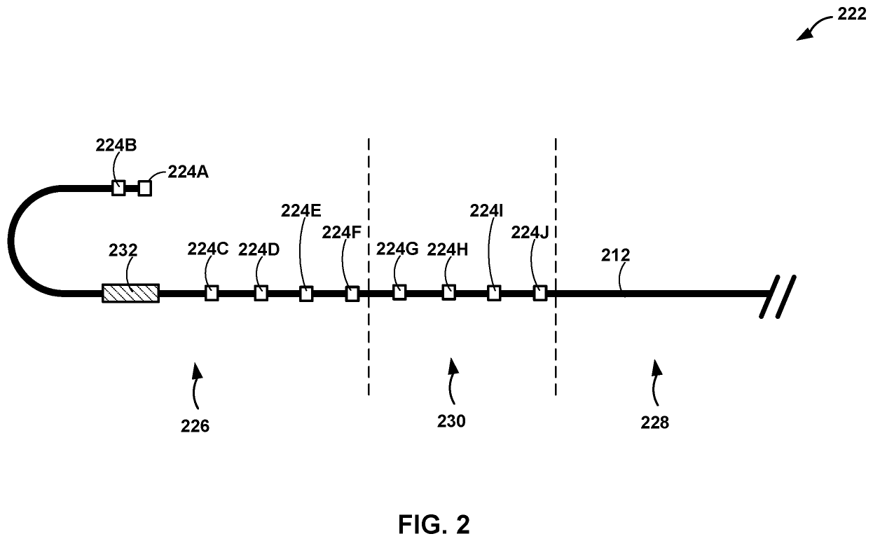

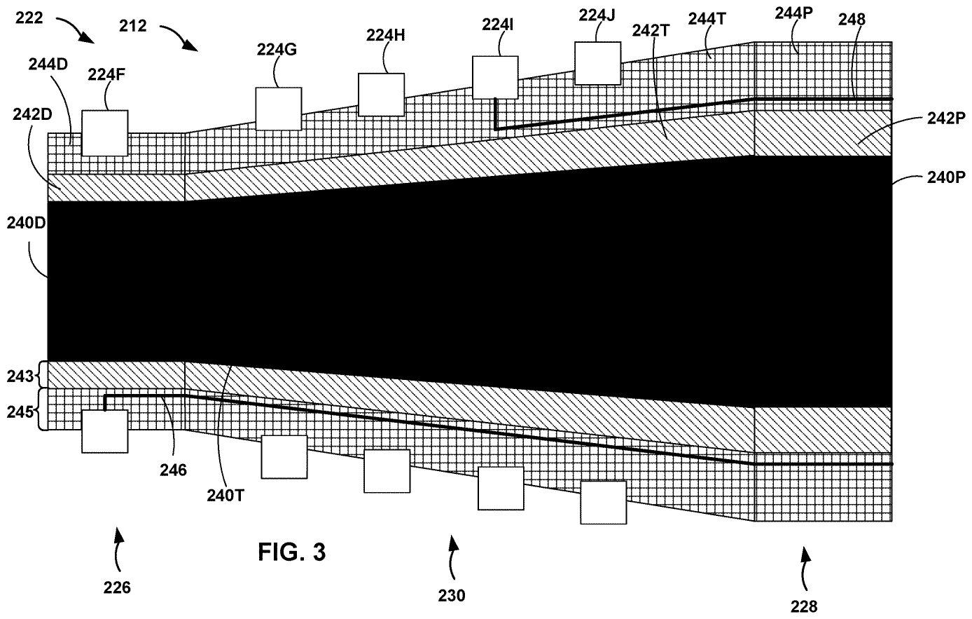

In some examples, guidewire 122 may include one or more proximal electrical connectors (not shown) configured to provide electrical insulation, mechanical support, and reduce connector interference with the environment, e.g., catching or snagging on surgical drapes and/or other portions of the environment outside of the patient's body. For example, guidewire 122 may include a proximal electrical cable and/or connector as described below with reference to FIGS. 2 and 3 and guidewire 222.

Electrodes 124 may be of any suitable geometry. Example geometries of electrodes include, but are not necessarily limited to, circular (e.g., ring) electrodes surrounding the core member of guidewire 122, conformable electrodes, cuff electrodes, segmented electrodes (e.g., electrodes disposed at different circumferential positions around guidewire 122 instead of a continuous ring electrode), or any combination thereof (e.g., ring electrodes and segmented electrodes). Electrodes 124 may be axially distributed along longitudinal axis LA of guidewire 122. Electrodes 124 may also be in a circular form, in an array, along multiple splines, or in other configurations.

Guidewire 122 may include signal wires, e.g., conductors, configured to carry electrical signals between electrodes 124 and controller 104. In some examples, guidewire 122 may include a separate signal wire for each of electrodes 124. For instance, in the example of FIG. 1 where electrodes 124 includes two electrodes, guidewire 122 may include two separate conductors. In this way, guidewire 122 may enable each electrode of electrodes 124 to be driven with a different signal from controller 104 and/or enable controller 104 to acquire different signals sensed by different electrodes 124. In other examples, multiple electrodes of electrodes 124 may share a common conductor. For instance, electrodes 124A and 124B may be connected to a same (e.g., a common) conductor. While such a common conductor arrangement may reduce energy delivery element flexibility (e.g., as electrodes connected to the common conductor may be driven with a same signal), such an arrangement may reduce manufacturing complexity and/or cost and may increase the structural flexibility of guidewire 122.

As shown in FIG. 1, electrodes 124 may include a tip electrode (e.g., electrode 124A), which may be a ring electrode with a “cap” covering at least a portion of a tip of guidewire 122. In some examples, the tip electrode may be chamfered or otherwise rounded (e.g., to enable easier passage of guidewire 122 through anatomy of the patient). Electrodes 124 may include a ring electrode (e.g., electrode 124B) that is adjacent to the tip electrode. This ring electrode may be separated (axially along LA) from the tip electrode. Electrodes 124 may include one or more pairs of ring electrodes (not shown). A pair of ring electrodes may include two adjacently closely spaced electrodes of electrodes 124. In general, one or more pairs of ring electrodes of guidewire 122 may be accompanied by one or more additional electrodes. The one or more additional electrodes may include any combination of pairs of ring electrodes and coil electrodes (e.g., electrodes that include conductors that spiral around guidewire 122).

In the example of FIG. 1, energy electrodes 124 are illustrated as has having a larger diameter than guidewire 122. In some examples, one or more of electrodes 124 may have a diameter that is approximately equal to or less than the diameter of guidewire 122. For instance, electrodes 124 may be recessed in guidewire 122 such that the combination results in a relatively smooth outer surface.

Controller 104 may include an energy generator configured to provide electrical pulses to electrodes 124 (or to control the delivery of radio frequency energy or cryogenic energy via electrodes 124 configured to be radio frequency delivery elements or cryogenic energy delivery elements) to perform an ablation procedure to cardiac tissue or other tissues within the patient's body, such as renal tissue, airway tissue, and organs or tissue within the cardiac space or the pericardial space. For instance, the energy generator may be configured and programmed to deliver pulsed, high-voltage electric fields appropriate for achieving desired pulsed, high-voltage ablation, e.g., PFA, “pulsed electric field ablation,” and/or pulsed radiofrequency ablation, albeit at voltages that may be less than those of energy delivery elements 110. In some examples, the energy generator may be configured and programmed for achieving desired cryogenic ablation. In some examples, controller 104 may include circuitry configured to receive and/or acquire electrical signals sensed and/or received by electrodes 124, e.g., EGM signals, or the like.

In some example, guidewire 122 may have a stiffness and/or flexibility that varies along its longitudinal length. For example, distal portion 126 may be more flexible to atraumatically navigate within the patient, e.g., the distal portion 126 may be a “floppy tip” having substantial flexibility. Proximal portion 128 may be more stiff to improve the manipulability of the guidewire, e.g., by a clinician. In some examples, guidewire 122 may include a transition portion (shown in FIG. 2) between the proximal portion 128 and distal portion 126 having a stiffness and/or flexibility between that of the proximal portion 128 and distal portion 126. The transition region may be configured to provide a smooth stiffness transition between the stiffnesses and/or flexibilities of the proximal portion 128 and distal portion 126, e.g., to reduce and/or eliminate kinking of guidewire 122.

The techniques of this disclosure may provide improved delivery of energy, such as PFA energy, using a high voltage PFA catheter. For example, guidewire 122 may be configured to reduce and/or prevent short circuiting of high voltage between adjacent PFA electrodes while maintaining a flexible, atraumatic, navigable distal portion and/or tip. The techniques of this disclosure may also improve delivery of PFA energy by providing additional energy delivering elements (e.g., electrodes 124) on guidewire 122 which may enable independent delivery of energy with different amounts and/or amplitudes relative to PFA catheter 102. Additionally, the techniques of this disclosure may also improve navigation of the guidewire 122 by providing electrodes 124 which may be configured to be radiopaque and/or electrical current and/or electrical field sensors usable to determine the positioning of distal portion 126 based on imaging and/or electrical responses of patient anatomy.

FIG. 2 is a conceptual diagram illustrating an example guidewire 222 for guiding an ablation catheter, e.g., PFA catheter 102. Guidewire 222 includes elongated member 212, electrodes 224A-224J (collectively, “electrodes 224”), and optionally navigation coil 232. Guidewire 222 may be substantially similar to guidewire 122 described above, e.g., elongated member 212 may be configured to be inserted into a patient to a target tissue site and to guide catheter 102 to the target tissue site and may comprise a core member encapsulated within an electrical insulator configured to electrically insulate the core member from electrical energy, such as PFA energy, delivered by energy delivery elements 110.

In the example shown, guidewire 122 includes distal portion 226, proximal portion 228, and transition portion 230, each of which may be substantially similar to proximal portion 126, distal portion 128, and the transition portion, respectively, described above. Electrodes 224 maybe substantially similar to electrodes 124 described above. In the example shown, guidewire 222 illustrates an example distribution of electrodes 124 along distal portion 226 and transition portion 230. Additionally, distal portion 226 may be a floppy tip and/or “J-tip” distal portion.

In the example shown, distal portion 226 includes tip electrode 224A, electrode 224B (which may be an electrode pair 224A-224B), and electrodes 224C-224F. Transition portion 230 includes electrodes 224G-224J, and proximal portion 228 includes no electrodes. In other examples, distal portion 226, transition portion 230, and proximal portion 228 may include a different proportion of the number of electrodes 224. Distal portion 226 optionally includes navigation coil 232.

In some examples, distal portion 226 is substantially flexible and/or a “floppy tip,” and transition portion 230 tapers in stiffness from the stiffness of distal portion 226 to a greater stiffness of proximal portion 228 (e.g., such that the transition portion tapers in stiffness from a first stiffness to a second stiffness). In some example, the thicknesses and/or materials may differ between distal portion 226, proximal portion 228, and transition portion 230. For example, the thicknesses of the core member, insulation, and/or signal wires of distal portion 226 may be less than that of proximal portion 228, and the thicknesses may increase within transition portion 230 from distal portion 226 to proximal portion 228. Additionally or alternatively, proximal portion 228 may include a stiffening structure while distal portion 226 does not, which transition portion 230 including a stiffening structure that tapers between distal portion 226 and proximal portion 228. In some examples, distal portion 226 may include a stiffening structure that is thinner and/or less stiff than a stiffening structure of proximal portion 228, and transition portion 230 may include a stiffening structure that tapers in stiffness between the stiffening structures of distal portion 226 and proximal portion 228. In some examples, transition portion 230 may extend from approximately 5 centimeters (cm) to 7 cm, or approximately 8 cm, from the distal tip of guidewire 222.

In some examples, the useable portion of guidewire 222 is approximately 170 cm to 210 cm, and the outer diameter of guidewire 222 is approximately 0.7 mm to 1.0 mm (including electrodes), and the radius of the “J-tip′ is approximately 2 mm to 4 mm. In some examples, electrodes 224 may be platinum-iridium and have a length of about 0.5 mm to 4.0 mm. In some examples, tip electrode 224A and/or 224B may be a metal ball at the distal tip of guidewire 222. In some examples, electrodes 224C-224J may have an edge-to-edge spacing of about 5.0 mm, or about 3.0 mm to about 10.0 mm.

FIG. 3 is a cross sectional view of a portion of the example guidewire 222 of FIG. 2. In the example shown, guidewire 222 includes core member 240, electrical insulator 242, electrodes 224, signal wires 246, 248, and electrically insulating jacket 244.

Core member 240 may include a proximal portion 240P, distal portion 240D, and transition portion 240T (e.g., collectively “core member 240”). Core member proximal portion 240P may have a first stiffness, core member distal portion 240D may have a second stiffness that is less than the first stiffness, and core member transition portion 240T may have a stiffness that tapers in stiffness from the first stiffness to the second stiffness along its longitudinal (e.g., axial) length. In this way, guidewire 222 may have a stiffness that varies correspondingly, e.g., proximal portion 228 may have a first guidewire 222 stiffness, distal portion 226 may have a second guidewire 222 stiffness, and transition portion 230 may have a guidewire 222 stiffness that tapers in stiffness from the first guidewire 222 stiffness to the second guidewire 222 stiffness. In other words, the stiffness of guidewire 222 may correspond to the stiffness of core member 240, with other components and or structures (e.g., electrical insulator 242, signal wires 246, 248, electrically insulating jacket 244, any polymeric stiffening structures, and electrodes 224) contributing to the overall stiffness of guidewire 222, albeit in some examples to a lesser degree.

In the example shown, the first stiffness, second stiffness, and transition stiffnesses of core member 240 correspond to the thicknesses of proximal portion 240P, distal portion 240D, and transition portion 240T, e.g., core member proximal portion 240P has a greater thickness and/or diameter than core member distal portion 240D, and core member transition portion 240T changes in thickness between the two. In other examples, each of proximal portion 240P, distal portion 240D, and transition portion 240T may have the same thickness, and the stiffness of the materials used may cause core member proximal portion 240P to have a greater stiffness that core member distal portion 240D, with core member transition portion 240T varying in stiffness between the stiffnesses of core member proximal portion 240P and core member distal portion 240D. In some examples, each of proximal portion 240P, distal portion 240D, and transition portion 240T may have the same thickness, and the stiffness and/or materials of any of core member 240, electrical insulator 242, electrodes 224, signal wires 246, 248, and/or electrically insulating jacket 244 may alone, or any combination, cause guidewire 222 to have a greater stiffness at the proximal portion than the transition portion, and a greater stiffness at the transition portion than the distal portion of guidewire 222. In some examples, the stiffness of guidewire 222 may be varied along its length by varying the durometer of any of core member 240, electrical insulator 242, and/or electrically insulating jacket 244.

Core member 240 may comprise stainless steel, Nitinol, a polymeric structure, and/or any material suitable as a guidewire core. In some examples, core member 240 includes a polymeric stiffening structure. For example, core member 240 may include at least one of polyether ether ketone (PEEK), a polyimide and/or polyamic acid such as PYRE-M.L., or the like. In some examples, the different stiffnesses of proximal portion 240P, distal portion 240D, and transition portion 240T are due to different amounts, types, and/or thicknesses of the polymeric stiffening structure. In some examples, electrically insulating jacket 244 may comprise the polymeric stiffening structure and may encapsulate at least a portion of a signal wire, e.g., signal wires 246, 248. In other examples, the polymeric stiffening structure may be separate from electrically insulating jacket 244 and may encapsulate at least a portion of a signal wire. In some examples, the polymeric stiffening structure, core member 240, electrical insulator 242, and/or insulating jacket 244 may be configured to provide improved and/or specific handling characteristics, e.g., at least a threshold thickness, friction, and/or grippability and/or manipulability.

Electrical insulator 242 may include a proximal portion 242P, distal portion 242D, and transition portion 242T (e.g., collectively “electrical insulator 242”). In some examples, electrical insulator 242 may vary in thickness, e.g., corresponding to the varying stiffness of core member 240. For example, electrical insulator proximal portion 242P may be thicker than electrical insulator distal portion 242D, and electrical insulator transition portion 242T may vary in thickness between the thicknesses of the proximal portion 242P and distal portion 242D. In this way, electrical insulator 242 may contribute to the varying stiffnesses of guidewire 222, e.g., between distal portion 226, proximal portion 228, and transition portion 230. In other examples, proximal portion 242P, distal portion 242D, and transition portion 242T may have substantially the same thickness, and in still other examples, proximal portion 242P, distal portion 242D, and transition portion 242T may vary oppositely in thickness, e.g., electrical insulator proximal portion 242P may be thinner than electrical insulator distal portion 242D, and electrical insulator transition portion 242T may vary in thickness between the thicknesses of the proximal portion 242P and distal portion 242D. In other words, core member 240 may be the primary driver of the stiffness of guidewire 222, and electrical insulator 242 may vary in thickness independently of the desired stiffness of distal portion 226, proximal portion 228, and transition portion 230.

Electrical insulator 242 may be any material suitable for insertion into a patient and to electrically isolate core member 240 from PFA energy delivered by ablation catheter 102 having a voltage of at least 1,500 volts, or at least 2,000 volts, or at least 3,000 volts, or at least 4,000 volts, or at least 6,000 volts, or at least 8,000 volts, e.g., and electrical insulator 242 may be configured to do so without dielectric breakdown of electrical insulator 242. In some examples, electrical insulator 242 may be configured electrically isolate core member 240 from PFA energy delivered by ablation catheter 102 having a voltage of at least 1,500 volts (or at least 2,000 volts, or at least 3,000 volts, or at least 4,000 volts, or at least 6,000 volts, or at least 8,000 volts) in conjunction with electrically insulating jacket 244, e.g., each of electrical insulator 242 and electrically insulating jacket 244 may not be configured to electrically isolate core member 240 from at least 1,500 volts, but are configured to electrically isolate core member 240 from at least 1,500 volts in combination, e.g., when electrically insulating jacket 244 surrounds electrical insulator 242. In some examples, electrical insulator 242 may comprise parylene, a polyimide and/or polyamic acid such as PYRE-M.L., or any suitable electrical insulating material. In some examples, insulator 242 may have a thickness 243 of between about 1 micrometer and 500 micrometers, between about 10 micrometers and 50 micrometers, or between about 10 micrometers and about 30 micrometers. In some examples, jacket 244 may have a thickness 245 of between about 1 micrometer and 500 micrometers, between about 10 micrometers and 50 micrometers, or between about 10 micrometers and about 30 micrometers.

Signal wires 246, 248 may be configured to be electrically connected to an electrode of electrodes 224. In the example shown, signal wire 246 is electrically connected to electrode 224F, and signal wire 248 is electrically connected to electrode 224I. Guidewire 222 may include fewer or more signal wires, for example, signal wires configured to be electrically connected to electrodes 224G, 224H, and 224I may be included and at different circumferential positions about guidewire 222 (and thus not visible in the cross-section of FIG. 3), or all of electrodes 224 may be configured to be electrically connected to a single signal wire. In some example, core 240 may be configured to electrically connect to one or more of electrodes 224 and to function as a signal wire. Generally, signal wires 246, 248 comprise an electrically conductive material. In some examples, core member 240 may include and an electrically conductive material and may be configured to be electrically connected to an electrode of electrodes 224.

Signal wires 246, 248, and/or core 240 may be configured to be connected to a signal and/or power source at proximal portion 228. For example, proximal portion 228 of guidewire 222 may be configured to be connected to controller 104, e.g., signal wires 246, 248, and/or core 240 may be connected to controller 104 via a single and/or multiple conductor connection cable and/or connector (not shown). For example, system 100 may include guidewire 222 and a proximal connection cable and/or connector configured to be slid over a portion of proximal portion 228 of guidewire 222 and electrically connect and/or engage with signal wires 246, 248, and/or core 240 to connect one or more of electrodes 224 and/or navigation coil 232 to a signal or power source, e.g., controller 104. In some examples, the proximal connector may be configured to provide electrical insulation and/or isolation as well as mechanical insulation, isolation, and/or support of proximal termination points or ends of signal wires 246, 248, and/or core 240, e.g., to insulate and cover the proximal ends of signal wires 246, 248, and/or core 240. In some examples, the proximal connector may be a permanently connected electrical cable or connector, or the proximal connector may be a detachable electrical cable or connector. In some examples, electrodes 224 and/or navigation coil 232 may be configured to be wireless connected to a power and/or signal source, e.g., via a wireless power and/or signal interface.

Electrically insulating jacket 244 may include a proximal portion 244P, distal portion 244D, and transition portion 244T (e.g., collectively “electrically insulating jacket 244”). In some examples, electrically insulating jacket 244 may vary in thickness, e.g., corresponding to the varying stiffness of core member 240. For example, electrically insulating jacket proximal portion 244P may be thicker than electrically insulating jacket distal portion 244D, and electrically insulating jacket transition portion 244T may vary in thickness between the thicknesses of the proximal portion 244P and distal portion 244D. In this way, electrically insulating jacket 244 may contribute to the varying stiffnesses of guidewire 222, e.g., between distal portion 226, proximal portion 228, and transition portion 230. In other examples, proximal portion 244P, distal portion 244D, and transition portion 244T may have substantially the same thickness, and in still other examples, proximal portion 244P, distal portion 244D, and transition portion 244T may vary oppositely in thickness, e.g., electrical insulator proximal portion 244P may be thinner than electrical insulator distal portion 244D, and electrical insulator transition portion 244T may vary in thickness between the thicknesses of the proximal portion 244P and distal portion 244D. In other words, core member 240 may be the primary driver of the stiffness of guidewire 222, and electrically insulating jacket 244 may vary in thickness independently of the desired stiffness of distal portion 226, proximal portion 228, and transition portion 230. Electrically insulating jacket 244 may surround electrical insulator 242 and may be configured to house signal wires 246, 248.

Electrically insulating jacket 244 may be any material suitable for insertion into a patient and to electrically isolate signal wires 246, 248 from PFA energy delivered by ablation catheter 102 having a voltage of at least 1,500 volts, or at least 2,000 volts, or at least 3,000 volts, or at least 4,000 volts, or at least 6,000 volts, or at least 8,000 volts, e.g., and insulating jacket 244 may be configured to do so without dielectric breakdown of insulating jacket 244. In some examples, electrical insulator 244 may comprise at least one of a fluorocarbon polymer, a polytetrafluoroethylene (PTFE), a ethylene tetrafluoroethylene (ETFE), parylene, a polyimide and/or polyamic acid such as PYRE-M.L., or any suitable electrically insulating material.

Electrodes 224 are positioned along distal portion 226 and transition portion 230, as shown in FIGS. 2 and 3. In some examples, one or more of electrodes 224 is configured to sense an intracardiac electrocardiogram (ECG) signal, deliver a high voltage pulsed electric field, and/or is radiopaque. In some examples, one or more of electrodes 224 may comprise a modified surface. For example, one or more of electrodes 224 may comprise a high surface area conductive surface such as titanium nitride or tantalum nitride.

Generally, each of electrodes 224 (e.g., electrodes 224A-224J illustrated in FIGS. 2 and 3) is configured to not short energy delivery elements 110. For example, the longitudinal length of electrodes 224 may be less than a distance between a first PFA electrode disposed on an outer surface of the PFA catheter, e.g., energy delivery element 110B, and a second, adjacent PFA electrode disposed on the outer surface of the PFA catheter, e.g., energy delivery element 110C, that may have a different electrical potential. For example, shorting an electrode pair such as 110A, 110B, or 110C, 110D may not be a problem because the elements/electrodes may have the same electrical potential and/or be at the same voltage. In other words, PFA electrode pairs may already be electrically connected and effectively shorted, however, shorting or electrically connecting adjacent energy delivery elements 110 may be undesirable. Electrodes 224 may be configured to not short adjacent energy delivery elements 110 having different voltages and/or electrical potentials, e.g., by having a longitudinal length that is less than the distance between such adjacent energy delivery elements 110. In this way, if a portion of distal portion 226 of guidewire 222 folds back on itself and/or PFA catheter 102, guidewire 222 is configured to prevent shorting of the PFA electrodes.

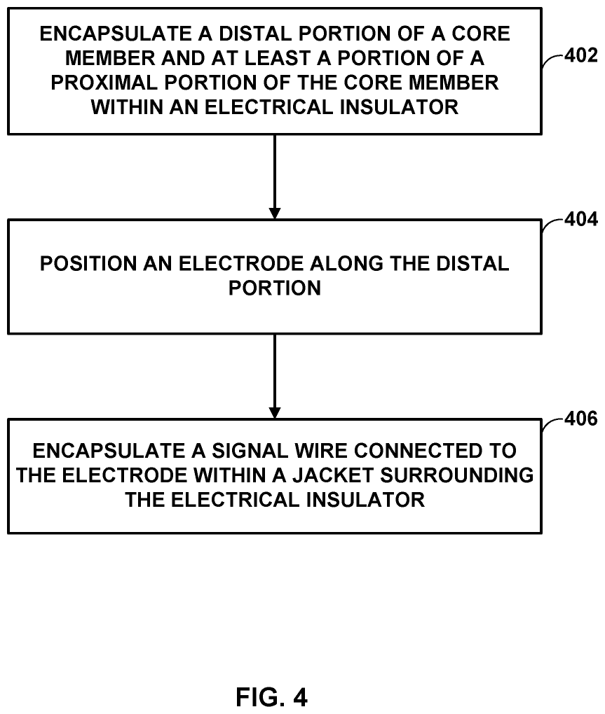

FIG. 4 is a flow diagram illustrating an example method of forming a guidewire 222 for guiding an ablation catheter 102. FIG. 4 is described with respect to guidewire 222 of FIGS. 2 and 3, however, the techniques of FIG. 4 may be utilized to make different guidewires.

A manufacturer may encapsulate a distal portion 240D of a core member 240 and at least a portion of a proximal portion 240P of the core member 240 within electrical insulator 242 comprising a material configured to electrically insulate the core member from pulsed field ablation (PFA) energy delivered by a PFA catheter (402). The manufacturer may then position an electrode 224 along the distal portion 226 (404). In some examples, the manufacturer may position one or more electrodes along the distal portion 226 and transition portion 230 of guidewire 222, and then electrically connect electrodes 224 to controller 104 via one or more signal wires, e.g., signal wires 246, 248. The manufacturer may then encapsulate a signal wire, or a plurality of signal wires 246, 248 within a jacket, e.g., electrically insulating jacket 244 surrounding the electrical insulator 242 (406). In some examples, the manufacturer may encapsulate core member 240, electrical insulator 242, and signal wires 246, 248 within electrically insulating jacket 244 with signal wires 246, 248 between electrical insulator 242 and electrically insulating jacket 244.

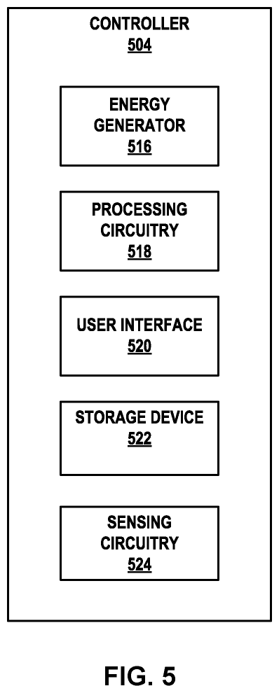

FIG. 5 is a block diagram illustrating an example controller of a multi-mode PFA system, in accordance with one or more aspects of this disclosure. Controller 504 of FIG. 5 may be an example of controller 104 of FIG. 1. As shown in FIG. 5, controller 504 may include energy generator 516, processing circuitry 518, user interface 520, storage devices 522, and sensing circuitry 524.

Energy generator 516 may be configured to provide electrical pulses to energy delivery elements and/or electrodes (e.g., energy delivery elements 110 of FIG. 1 and/or electrodes 224 of FIGS. 2 and 3) to perform an electroporation procedure to cardiac tissue or other tissues within the patient's body, such as renal tissue, airway tissue, and organs or tissue within the cardiac space or the pericardial space. For instance, energy generator 516 may be configured and programmed to deliver pulsed, high-voltage electric fields appropriate for achieving desired pulsed, high-voltage ablation (referred to as “pulsed field ablation” or “pulsed electric field ablation”) and/or pulsed radiofrequency ablation. While shown in the example of FIG. 5 as a single energy generator, energy generator 516 is not so limited. For instance, controller 504 may include multiple energy generators that are each capable of generating ablation signals in parallel.

Processing circuitry 518 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 518 herein may be embodied as firmware, hardware, software or any combination thereof. Processing circuitry 518 controls energy generator 516 to generate signals according to various settings (e.g., linear settings 530 or focal settings 532). In some examples, processing circuitry 518 may execute other instructions stored in storage device 522 to perform PFA.

Sensing circuitry 524 may be configured to receive signals from energy delivery elements 110 and/or 224. For example, electrodes 224 may be configured to sense EGM signals and sensing circuitry 524 may be configured to receive the ECG signals from one or more electrodes 224.

Storage device 522 may be configured to store information within controller 504, respectively, during operation. Storage device 522 may include a computer-readable storage medium or computer-readable storage device. In some examples, storage device 522 includes one or more of a short-term memory or a long-term memory. Storage device 522 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 522 is used to store data indicative of instructions, e.g., for execution by processing circuitry 518, respectively.

User interface 520 may include a button or keypad, lights, a speaker for voice commands, a display, such as a liquid crystal (LCD), light-emitting diode (LED). or organic light-emitting diode (OLED). In some examples, the display includes a touch screen. User interface 520 may be configured to display any information related to the performance of PFA. User interface 520 may also receive user input (e.g., selection of linear or focal PFA mode) via user interface 520. The user input may be, for example, in the form of pressing a button on a keypad or selecting an icon from a touch screen.

Accordingly, although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1. A guidewire comprising: a core member comprising: a proximal portion having a first stiffness; and a distal portion having a second stiffness less than the first stiffness; an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter guided by the guidewire; and an electrode positioned along the distal portion.

Example 2. The guidewire of claim 1, wherein the core member further comprises: a transition portion between the proximal portion and the distal portion, wherein the transition portion tapers in stiffness from the first stiffness to the second stiffness.

Example 3. The guidewire of claim 2, wherein the guidewire further comprises a second electrode positioned along the transition portion.

Example 4. The guidewire of claim 1 or claim 2, further comprising: a jacket surrounding the electrical insulator and configured to house a signal wire, wherein the signal wire is configured to be electrically connected to the electrode.

Example 5. The guidewire of claim 4, wherein the electrical insulator is comprised of parylene, wherein the jacket is comprises of at least one of polytetrafluoroethylene (PTFE) or ethylene tetrafluoroethylene (ETFE).

Example 6. The guidewire of any one of claims 1-5, wherein the electrical insulator is configured to electrically isolate the core member from PFA energy delivered by the PFA catheter having a voltage of at least 1,500 volts.

Example 7. The guidewire of any one of claims 1-6, further comprising a navigation coil positioned along the distal portion.

Example 8. The guidewire of any one of claims 1-7, wherein the electrode is configured to sense an intracardiac electrocardiogram (ECG) signal.

Example 9. The guidewire of any one of claims 1-8, wherein the electrode is radiopaque.

Example 10. The guidewire of any one of claims 1-9, wherein the electrode is configured to deliver a high voltage pulsed electric field, and wherein a surface of the electrode comprises a high surface area conductive surface comprising at least one of titanium nitride or tantalum nitride.

Example 11. The guidewire of any one of claims 1-10, wherein the core member comprises a polymeric stiffening structure.

Example 12. The guidewire of claim 11, wherein the polymeric stiffening structure comprises at least one of a polyether ether ketone (PEEK) or a polyimide.

Example 13. The guidewire of claim 12, wherein the polymeric stiffening structure encapsulates at least a portion of a signal wire, wherein the signal wire is configured to be electrically connected to the electrode.

Example 14. The guidewire of any one of claims 1-13, wherein a length of the electrode along a longitudinal axis of the guidewire is less than a distance between a first PFA electrode disposed on an outer surface of the PFA catheter and a second PFA electrode disposed on the outer surface of the PFA catheter, wherein the second PFA electrode is adjacent the first PFA electrode, wherein the first PFA electrode has a first electrical potential and the second PFA electrode has a second electrical potential different form the first electrical potential.

Example 15. A medical system comprising: a pulsed field ablation (PFA) catheter; and a guidewire comprising: a core member comprising: a proximal portion having a first stiffness; and a distal portion having a second stiffness less than the first stiffness; an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter; and an electrode positioned along the distal portion.

Example 16. The medical system of claim 15, wherein the core member further comprises: a transition portion between the proximal portion and the distal portion, wherein the transition portion tapers in stiffness from the first stiffness to the second stiffness.

Example 17. The medical system of claim 16, wherein the guidewire further comprises a second electrode positioned along the transition portion.

Example 18. The medical system of any one of claims 15-17, further comprising: a jacket surrounding the electrical insulator and configured to house a signal wire, wherein the signal wire is configured to be electrically connected to the electrode.

Example 19. The medical system of claim 18, wherein the electrical insulator is comprised of parylene, wherein the jacket is comprises of at least one of polytetrafluoroethylene (PTFE) or ethylene tetrafluoroethylene (ETFE).

Example 20. The medical system of any one of claims 15-19, wherein the electrical insulator is configured to electrically isolate the core member from PFA energy delivered by the ablation catheter having a voltage of at least 1,500 volts.

Example 21. The medical system of any one of claims 15-20, further comprising a navigation coil positioned along the distal portion.

Example 22. The medical system of any one of claims 15-21, wherein the electrode is configured to sense an intracardiac electrocardiogram (ECG) signal.

Example 23. The medical system of any one of claims 15-22, wherein the electrode is radiopaque.

Example 24. The medical system of any one of claims 15-23, wherein the electrode is configured to deliver a high voltage pulsed electric field.

Example 25. The medical system of any one of claims 15-24, wherein the core member comprises a polymeric stiffening structure.

Example 26. The medical system of claim 25, wherein the polymeric stiffening structure comprises polyether ether ketone (PEEK).

Example 27. The medical system of claim 25 or claim 26, wherein the polymeric stiffening structure encapsulates at least a portion of a signal wire, wherein the signal wire is configured to be electrically connected to the electrode.

Example 28. The medical system of any one of claims 15-27, wherein a length of the electrode along a longitudinal axis of the guidewire is less than a distance between a first PFA electrode disposed on an outer surface of the PFA catheter and a second PFA electrode disposed on the outer surface of the PFA catheter, wherein the second PFA electrode is adjacent the first PFA electrode, wherein the first PFA electrode has a first electrical potential and the second PFA electrode has a second electrical potential different form the first electrical potential.

Example 29. The medical system of any one of claims 15-28, wherein the core member comprises an electrically conductive material and is configured to be electrically connected to the electrode.

Example 30. A method of forming a guidewire comprising: encapsulating a distal portion of a core member and at least a portion of a proximal portion of the core member within an electrical insulator, wherein the electrical insulator comprises a material configured to electrically insulate the core member from pulsed field ablation (PFA) energy delivered by a PFA catheter, wherein the proximal portion of the core member has a first stiffness and the distal portion of the core member has a second stiffness less than the first stiffness; and positioning an electrode along the distal portion.

Example 31. The method of claim 30, further comprising: encapsulating a signal wire within a jacket, the jacket surrounding the electrical insulator, wherein the signal wire is between the electrical insulator and jacket, wherein the signal wire is electrically connected to the electrode.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof, Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

1. A guidewire comprising:
a core member comprising:
a proximal portion having a first stiffness; and
a distal portion having a second stiffness less than the first stiffness;
an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter guided by the guidewire; and
an electrode positioned along the distal portion.

2. The guidewire of claim 1, wherein the core member further comprises:
a transition portion between the proximal portion and the distal portion, wherein the transition portion tapers in stiffness from the first stiffness to the second stiffness.

3. The guidewire of claim 2, wherein the guidewire further comprises a second electrode positioned along the transition portion.

4. The guidewire of claim 1, further comprising:
an electrically insulating jacket surrounding the electrical insulator and configured to house a signal wire, wherein the signal wire is configured to be electrically connected to the electrode.

5. The guidewire of claim 4, wherein the electrical insulator is comprised of parylene, wherein the electrically insulating jacket is comprises of at least one of polytetrafluoroethylene (PTFE) or ethylene tetrafluoroethylene (ETFE).

6. The guidewire of claim 1, wherein the electrical insulator is configured to electrically isolate the core member from PFA energy delivered by the PFA catheter having a voltage of at least 1,500 volts.

7. The guidewire of claim 1, further comprising a navigation coil positioned along the distal portion.

8. The guidewire of claim 1, wherein the electrode is configured to sense an intracardiac electrocardiogram (ECG) signal.

9. The guidewire of claim 1, wherein the electrode is radiopaque.

10. The guidewire of claim 1, wherein the electrode is configured to deliver a high voltage pulsed electric field, and wherein a surface of the electrode comprises a high surface area conductive surface comprising at least one of titanium nitride or tantalum nitride.

11. The guidewire of claim 1, wherein the core member comprises a polymeric stiffening structure.

12. The guidewire of claim 11, wherein the polymeric stiffening structure comprises at least one of a polyether ether ketone (PEEK) or a polyimide.

13. The guidewire of claim 12, wherein the polymeric stiffening structure encapsulates at least a portion of a signal wire, wherein the signal wire is configured to be electrically connected to the electrode.

14. The guidewire of claim 1, wherein a length of the electrode along a longitudinal axis of the guidewire is less than a distance between a first PFA electrode disposed on an outer surface of the PFA catheter and a second PFA electrode disposed on the outer surface of the PFA catheter, wherein the second PFA electrode is adjacent the first PFA electrode, wherein the first PFA electrode has a first electrical potential and the second PFA electrode has a second electrical potential different form the first electrical potential.

15. A medical system comprising:
a pulsed field ablation (PFA) catheter; and
a guidewire comprising:
a core member comprising:
a proximal portion having a first stiffness; and
a distal portion having a second stiffness less than the first stiffness;
an electrical insulator encapsulating the distal portion of the core member and at least a portion of the proximal portion of the core member, wherein the electrical insulator comprises a material configured to electrically insulate the core member from pulsed field ablation (PFA) energy delivered by a PFA catheter; and
an electrode positioned along the distal portion.

16. The medical system of claim 15, wherein the core member further comprises:
a transition portion between the proximal portion and the distal portion, wherein the transition portion tapers in stiffness from the first stiffness to the second stiffness.

17. The medical system of claim 16, wherein the guidewire further comprises a second electrode positioned along the transition portion.

18. The medical system of claim 15, further comprising:
an electrically insulating jacket surrounding the electrical insulator and configured to house a signal wire, wherein the signal wire is configured to be electrically connected to the electrode.

19. A method of forming a guidewire comprising:
encapsulating a distal portion of a core member and at least a portion of a proximal portion of the core member within an electrical insulator, wherein the electrical insulator comprises a material configured to electrically insulate the core member from pulsed field ablation (PFA) energy delivered by a PFA catheter, wherein the proximal portion of the core member has a first stiffness and the distal portion of the core member has a second stiffness less than the first stiffness; and
positioning an electrode along the distal portion.

20. The method of claim 19, further comprising:
encapsulating a signal wire within an electrically insulating jacket, the electrically insulating jacket surrounding the electrical insulator, wherein the signal wire is between the electrical insulator and the electrically insulating jacket, wherein the signal wire is electrically connected to the electrode.
